(19) United States
(12) Patent Application Publication
(10) Pub. No.: US 2010/0324365 A1
(43) Pub. Date: Dec. 23, 2010

(54) Diagnostic Capsule with Software that Triggers Imaging Equipment

(76) Inventor: Daniel Victor Marquez, (US)

Correspondence Address: DANIEL VICTOR MARQUEZ, 27117 WESTVIEW LANE, VALENCIA, CA, 91354, US

(21) Appl. No.: 12/486,712

(22) Filed: Jun. 17, 2009

(51) Int. Cl. A61B 1/04

(52) U.S. Cl. 600/109; 600/117

(57) ABSTRACT

Disclosed is a compact ingestible capsule that can transmit gut-diagnostic data to a computer. In a preferred embodiment, the capsule can transmit data such as enzyme concentration and pH, while a program generates triggers that can be used to activate imaging equipment to disclose the location of the capsule at a trigger event.

### BRIEF SUMMARY OF THE INVENTION

The invention provides a compact ingestible diagnostic capsule that can analyze the presence and concentration of gut analytes such as enzyme concentration, biotoxins, flora markers, parasite markers (such as parasite waste or toxins), food concentration, bile concentration, and more.

A preferred embodiment of the capsule uses at least one well that is embedded with material that changes at least one optical property in the presence of a specific analyte. For example, the well may be filled with an opaque fat that blocks a light created an LED within the capsule. When a lipase dissolves the fat, an optical sensor detects light from the LED.

A third embodiment of the capsule uses at least one hollow well to detect the presence of analyte within the surrounding fluid. This embodiment is ideal for characterizing the bioavailability of nutrients, supplements, and drugs.

A fourth embodiment consists of any combination of the aforementioned embodiments to analyze any combination of analytes.

Any embodiment may consist of a means to measure temperature, pH, pressure, and position to further enhance the usefulness of collected data.

Because of the simplicity and diversity of the embodiments, the invention may be used to characterize analytes in humans, animals, and chambers that contain fluids or gases.

### FIELD OF THE INVENTION

The present invention relates generally to a compact wireless diagnostic capsule which can be swallowed to collect data of an environment such as a digestive tract.

### BACKGROUND OF THE INVENTION

Research has shown that the root cause of many chronic diseases can be traced to diseases relating to the gut. For instance, low levels of elastase can trigger a proliferation of gram-negative bacteria such as Salmonella and E. Coli, which can trigger diarrhea, abdominal cramping, and infections of other parts of the body. When pathogens escape the digestive tract, they can translocate to any tissue and create an indefinite number of symptoms, including arthritis, asthma, rhinitis, backache, and skin rashes. In more severe cases, the central nervous system can become inflamed, leading to symptoms ranging from dementia to death.

Unfortunately, characterizing gut parameters can be complicated, time consuming, risky, and expensive. For instance, health professionals may incorporate the use of complex machines such as the ERCP (Endoscopic Retrograde Cholangiopancreatography). While these can be life saving, they require extensive training and the patients must be sedated with chemicals that can damage the liver or pancreas.

Because of these reasons, some health professionals prefer to use less invasive diagnostic capsules such as the Heidelberg and endoscopic camera capsules to diagnose gut problems. While the Heidelberg capsule is widely used to characterize gut pH, the camera capsule is generally used for locating tumors, inflammation, worms, and more. However, neither of these powerful tools characterizes digestive enzyme concentrations, a requirement to determining the cause of many diseases.

There are emerging diagnostic capsules that do characterize biological parameters within the gut, but they can be large and expensive, especially when they incorporate ultrasound transducers for capsule tracking.

Because of the complexity and expense of diagnostic tools, many health practitioners resort to the practice of “masking symptoms” rather than curing the diseases. For example, patients who complain of nausea may be given medications such as meclizine, amitriptyline, scopolamine, Benadryl, lorazepam, and metoclopramide. Sadly, these drugs merely reduce the symptoms, and do not address the root cause of the nausea.

The overwhelming number of diseases relating to the gut can overwhelm even the most astute health professionals, especially when they are unable to equip facilities with state-of-the-art gut diagnostic equipment because of cost, space constraints, and the lack of trained personnel. There is clearly a need for a diagnostic device that is cost effective, simple to use, and thorough with immediate diagnostic results.

### DESCRIPTION OF THE INVENTION

The present invention is a compact ingestible pill that consists of a plurality of aperture wells that contain substances that change optical properties in the presence of specific biochemical conditions (such as analyte concentration or pH). The invention includes an optical transmitter and corresponding sensor at each well, and a means to transmit measured optical parameters to a processing system. An analyte is defined as any enzyme, biomarker, hormone, protein, biotoxin, nutrient, antibody, poison, drug, or organism such as a bacterium or a white blood cell.

Reference is now made to FIG. 1. This illustrates a capsule 1 that has a plurality of wells 5. Each well 5, typically embedded with an analytical material such as food, exhibits optical properties that change in the presence of analytes within the surrounding fluid. For instance, to detect lipases, a well 5 may be embedded with fat. When the fat dissolves, the transmittance changes. In the case where the surrounding fluid is clear, the transmittance diminishes. The rate at which the transmittance diminishes would determine the potency of the lipase. Because there are different kinds of lipases, different kinds of fats may be used to detect each lipase.

Wells filled with protein can be used to characterize proteases. The presence of proteases will break down the protein, and will therefore affect the optical parameters of the protein. Because a specific protease breaks a specific amino acid bond, the ideal protein for a protease characterization would be a polypeptide made with a chain of the specific amino acid bonds. If a protein within a well fails to break down, that may be indicative of a low protease concentration if the pH is normal.

Wells embedded with starch, as another example, can be used to detect the presence of amylases. Because there are different kinds of amylases, different kinds of starches can be used to detect specific amylases. Because salivary amylases (AMY1A, AMY1B, and AMY1C) are generally neutralized by gastric acid, the pancreatic amylases (AMY2A and AMY2B) may be clinically more important. Calcium can be added to the starch sites to ensure that the alpha amylases are activated.

Reference is now made to FIG. 2, which illustrates a construction of capsule 1. The heart of the electrical processing is hybrid 8, which consists of light emitter 10 that transmits light 11. In this embodiment, reflector 3 reflects light to light pipe 4, which transmits light 11 to through wells 5. The light is sensed with optical sensors 9, and sensed data is transmitted via antenna 7.

Hybrid 6 can consist of an indefinite number of circuit topologies. Its core processor, for instance, can be a mixed signal microcontroller, a PSoC (Programmable System on Chip), or an ASIC (Application-Specific Integrated Circuit). The advantage of a PSoC is that each pin can be programmed to be an analog or digital input or output. A system on chip such as Texas Instruments CC1110 consists of an internal RF driver in addition to mixed-signal functionality. In addition, it can be programmed so that it can support any configuration of wells; gains, A-to-D conversion, timing pulses, RF frequency, modulation algorithms, and power management can be programmed with assembly or C.

Reference is now made to FIG. 3. This illustrates an embodiment of FIG. 1. In this embodiment, light sources 10a to 10e, wells 5a to 5e, light beams 11a to 11e, and light sensors 9a to 9e. This embodiment is ideal in that each well can have an independent light source and matching light detector. Unlike the previous embodiment of FIG. 2, no mirror or light pipe is required.

Reference is now made to FIG. 4, which illustrates a close-up of well 5. The well is embedded with material 15 that changes optical properties when exposed to analyte. Light beam traverses from LED 10 to optical sensor 9.

A variation of FIG. 4 is shown is shown in FIG. 5, which illustrates a material 15 that is enteric coated with a coating that protects the material from the gastric environment, and that dissolves in the intestines. Enteric coatings include pectins or polymeric carbohydrates such as alginates, and are frequently used to protect probiotics and certain medicines from harsh stomach acid.

There are a number of ways to measure optical parameters of well material 5. Reference is now made to FIG. 6a, which illustrates an optical sensor that employs LED 50 and phototransistor 800. In this embodiment, the photodiode is biased with a current source as an example. The phototransistor 800 forms a voltage divider with resistor R2. Light intensity varies the impedance of the phototransistor 800. When the light intensity is increased to a sufficient level, comparator 33 is triggered and processed by any digital processor such as a PSoC.

Reference is now made to FIG. 6b, which illustrates another embodiment of an optical sensor. In this example, a photodiode 801 is configured in a photoconductive mode, which increases a voltage at resistor R3 with increasing light. Amplifier 34 can be configured to buffer or amplify the voltage at R3. While FIG. 6b illustrates a photoconductive mode circuit, other modes such as photovoltaic mode and avalanche mode can also be incorporated.

Because an LED can consume a significant amount of current (for example, 2 mA) from the battery, it is often desirable to pulse the LED off to conserve power, and on just prior to an optical measurement. By incorporating pulse width modulation, the diode can be brightly lit for brief pulses without draining the battery significantly. Typically, high brightness increases sensitivity.

Reference is now made to FIG. 7, which illustrates a PWM waveform 7 that is used to modulate transistor switch Q50. Current source 51 turns the LED on when Q50 is on, and is blocked when Q50 is turned off, effectively turning off the LED. It is an object of the invention to use PWM to bias the LED's or other light sources.

To conserve energy even further, the detection circuitry can be shut off once desired data is collected. This can be done by programming the PSoC, by way of example, or by designing shutoff circuitry that's triggered when a desired threshold is seen. Those skilled in the art can implement a variety of methods to conserve energy, such as placing a CC1110 microcontroller in sleep mode until triggered to wake up.

Reference is now made to FIG. 8, which illustrates a luminous material 55, such as bioluminescence bacteria. In this embodiment, an LED is not needed, and a photodiode 800 is shown by way of example as a means to sense the bioluminescence. Bioluminescence can often be used to detect trace levels of analyte. For example, a lymphatic B-cell, which creates antibody, can be modified to glow when it senses a specific antigen.

To increase sensitivity to bioluminescence, reference is now made to FIG. 9. This illustrates a CCD (Charge Coupled Device) pixel used in sensitive cameras intended for low lighting. The most sensitive CCDs employ backlighting to increase quantum efficiency and to improve signal to noise ratio. Such a CCD is composed of three layers 881 (sensor area), 882 (on chip gain multiplication area), and 883 (on chip back illumination area). Like a photodiode, the CCD can be designed using any circuit mode.

Bioluminescence materials 55 are examples of a class of materials known as biosensors, materials used for detecting extremely low levels of analyte. Biosensors use biomediators to enhance sensitivity. The biomediators may include enzymes (e.g., light emitting luciferase), antibodies (to detect antigens), antigens (to detect antibodies), biological membranes, bacteria (natural or modified to become bioluminescent), immune cells, and tissue cells that interact directly or indirectly with the analyte. The interaction results in a measurable optical change such as a change in chemiluminescence or bioluminescence. There are many biosensors that can be used, and some would require enteric coating to be protected from the acidic gastric environment. Glowing biosensors may use the circuitry of FIGS. 8 and 9, while non-glowing biosensors may use circuits of FIGS. 3 to 7.

Reference is now made to FIG. 10a, which illustrates timing waveforms used to control the optical elements (the LED and the light sensor) of the preceding figures. Waveform 100 is an example of a digital waveform used to pulse the LED on and off. Waveform 101 is a waveform used to latch the voltage created by the light sensor. In this example, waveform 101 has a smaller width than waveform 100 so that data isn't captured before the LED settles. This timing is ideal for capturing data relating to transmittance or absorption.

Reference is now made to FIG. 10b, which illustrates the timing pulses for measuring fluorescence. In this example, fluorescence is measured after the LED is shutoff. This measurement is done with timing pulse 102, which effectively latches the ADC (Analog to Digital Converter), typically embedded in a PSoC. The exact timing of the displayed waveforms of FIG. 10a and FIG. 10b are merely examples of preferred embodiments, as there are many waveform patterns that can be used to enhance a measurement. For instance, waveform 102 can consist of multiple pulses to measure degradation of the fluorescence.

In many cases, it is important to know the position of a pill when certain optical properties are detected. For example, it is important to know where bleeding or an inflammation is occurring so that physicians can take corrective action. Many techniques have been implemented. U.S. Pat. No. 7,160,258, for instance, discloses a diagnostic capsule that is embedded with acoustic transducers used for precise tracking of the capsule within the gastrointentinal tract. Unfortunately, such transducers take up significant real estate and consume significant power. While it is possible to track the location of the capsule by triangulation of RF amplitudes detected outside the body, the location is rather crude.

It is the object of the current invention to acquire the capsule location at important time intervals or at important sense events without adding extra hardware to the capsule. One method is to program “image capture triggers” in the monitoring software, the software being used to monitor the data being transmitted from the capsule. The triggers can be used to trigger image capturing by existing equipment, such as an x-ray or such as an ultrasonic image. The triggers can also generate audible sound so that a medical technician can manually locate the pill with a handheld ultrasonic transducer, as an example, or with an x-ray. The advantage of triggering image captures is that no additional hardware is required for the capsule, and satisfactory resolution can be obtained. Furthermore, the capsule is very visible in both x-ray and ultrasonic images.

Because enzyme potency can be a function of pH, it is desirable to transmit pH values detected along the gut. Ideally, the pH monitor should be able to monitor pH values ranging from 1 to 9. It may be worth noting that enzymes residing in the stomach work best when the pH is low (between 1 to 3) while enzymes secreted by the pancreas work best in an alkaline environment, where pH levels are above 7.0. A deviation from these pH levels can have clinical significance. For example, if gastric pH is above 4, then it is possible that gall stones and pancreatitis could be triggered since a pH higher than 4 prevents the secretion of hormones secretin and CCK, the hormones that activate the release of bile and pancreatic enzymes. Any imbalance of the pH or pancreatic enzymes can trigger the pathogenic overgrowth. It is therefore desirable for a capsule to send pH data to determine a cause of enzyme inactivity.

Methods of measuring pH are well known, and many methods exit. The probe can be of the type indicated in U.S. Pat. Nos. 4,009,721, 6,689,056, and even U.S. Pat. No. 6,673,625, where fluorescence can indicate pH. The Heidelberg pH capsule is widely used by medical professionals to study a patient's pH profile.

Anthocyanins (found in berries, red cabbage, blood oranges, and colorful flowers such as red roses and geraniums) are excellent pH indicators. Anthocyanins are red in acidic environments and blue in alkaline environments. Furthermore, anthocyanins are believed to offer health benefits ranging from reducing urinary tract infections to reducing the growth of tumors.

Anthocyanins that are acylated with hydroxycinnamic acid are highly stable in the digestive tract. They can be stored in a translucent polysaccharide or porous stable material such as those disclosed in U.S. Pat. No. 7,335,514. Those skilled in the art of chemistry can determine a myriad of methods to hold anthocyanins in one location while allowing external digestive fluids to permeate.

We now refer to FIG. 11, which illustrates a method of detecting pH by observing color change of two identical wells (62a and 62b) that are made with two detectors (63a and 63b) and their respective amplifiers (64a and 64b). Each site, which contains pH indicator dye such as acylated anthocyanin, is illuminated with light source 60 and two color filters (61a and 61b). Filter 61a is a blue filter that blocks red light, and filter 61b is a red filter that blocks blue light. The pH is determined by comparing the blue light intensity against the red light intensity. The actual values, however, are determined by anthocyanin concentration, site construction, amplifier gain, light filter properties, and light source properties. Furthermore, the light detector 63a or 63b will be affected further if the measure light is reflected or transmitted. Those who are skilled in the art can make any similar table to detect color shift.

Using this method, two wells can be dedicated to measuring pH, while the remaining wells can be used to detect other parameters such as enzyme concentrations. The number of wells shown is simply by way of example. There can be one well or many wells, depending on what is being diagnosed and the circuitry that is being used.

Flora, or “friendly bacteria”, is an important of a healthy gut. Using a plurality of biosensors that can be quite costly. Therefore, detection of beta amylases is proposed. Unlike alpha amylases that humans create, beta amylases are created by intestinal microorganisms. Beta amylases can quickly cleave α-1,4-glycosidic bonds in calcium free starches. Thus, flora concentration can be measured with sites that are made with calcium-free starches that are made with α-1,4-glycosidic bonds. A rapid breakdown can suggest the presence of flora that aids in digestion. A slow breakdown would suggest the absence of such flora.

While there are many materials that resist breakdown during ingestion, the body of capsule 1 can be made of polyacrylate, a plastic used in ingestible capsules. To reduce the risk of biofilm overgrowth, small levels of silver powder can be added to the polyacrylate before it is hardened.

Temperature can also be used to detect the presence of infection, such as in the case of a localized fever. Temperature can be measured with diodes or thermistors that are embedded anywhere in the capsule. To maintain a simple mechanical design, the current embodiment uses a temperature sensor embedded in the capsule's processing circuit.

While the aforementioned embodiments are ideal for detecting analytes, they can also be used to detect the behavior of food extracts, medicines, probiotics, and chemicals that are embedded as analyte detectors.

The advantage of the diagnostic pill of FIG. 11 is that any material can be inserted in wells 103, and electrodes 8 and optical detectors 800 can be used to characterize the behavior of the material. The material can be used to detect analyte, or, alternatively, it can be used to characterize the behavior of a compound inside a fluid or gaseous environment. Therefore, the diagnostic capsule can be placed in an indefinite number of environments such as in chemical chambers and wine barrels, not just in the gut. Those skilled with chemistry can experiment with well materials for any kind of environment, and apply desired optical filters on the light sources or detectors.

One key advantage of a diagnostic pill of is that it can be used to characterize intestinal permeability, or “leaky gut” syndrome. By mapping the concentration of permeability markers (e.g., polysaccharides, lactulose, and mannitol) along the digestive tract (as can be done with the diagnostic pill), a diagnostician can determine the location and severity of a gut leaky. Currently, permeability markers are examined in urine samples or blood samples, but using a diagnostic capsule can provide the location of elevated permeability, a location typically associated with an infection.

After description of embodiments of the offered invention with the reference to applied drawings it is clear that the present invention is not limited by these embodiments. Obviously many modifications and variations of the present invention are possible in light of the above teachings. It is, therefore, to be understood that within the scope of the appended claims, the invention may be practiced otherwise than as specifically described.

### BRIEF DESCRIPTION OF THE DRAWINGS

FIG. 1 illustrates a diagnostic pill consisting of wells used to characterize analytes.

FIG. 2 illustrates the interior of one embodiment of FIG. 1.

FIG. 3 illustrates another embodiment of FIG. 1.

FIG. 4 illustrates an optical well with an embedded material used to characterize analytes.

FIG. 5 illustrates an optical well with an enteric coating used to protect embedded material from the harsh gastric environment.

FIG. 6a illustrates a circuit that shows a means of measuring light intensity across a well using a phototransistor.

FIG. 6b illustrates a circuit that shows a means of measuring light intensity across a well using a photodiode.

FIG. 7 illustrates a Pulse Width Modulation circuit that increases the brightness of a capsule's LED while decreasing the power consumption.

is a block diagram showing how the intensity of two colors can be used to measure pH.

FIG. 8 illustrates a means to detect low traces of analyte using a light-emitting biosensor.

FIG. 9 illustrates a means to detect extremely low levels of analyte using a light-emitting biosensor and a CCD chip that has backlighting and layered amplification.

FIG. 10a illustrates timing pulses used to measure transmittance across a well.

FIG. 10b illustrates timing pulses used to measure florescence of material within a well.

FIG. 11 illustrates a means to measure pH using the amplitudes of two wavelengths.

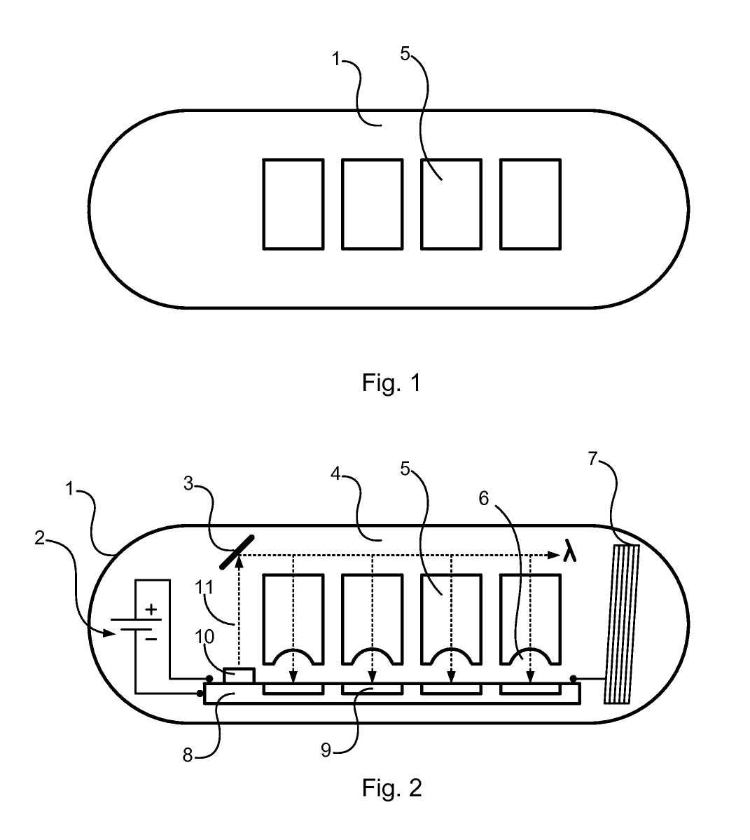

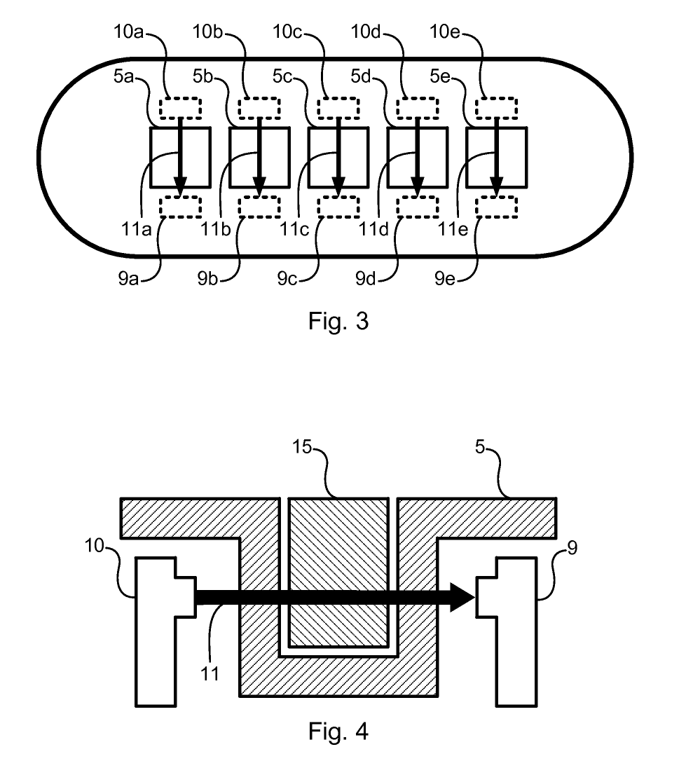

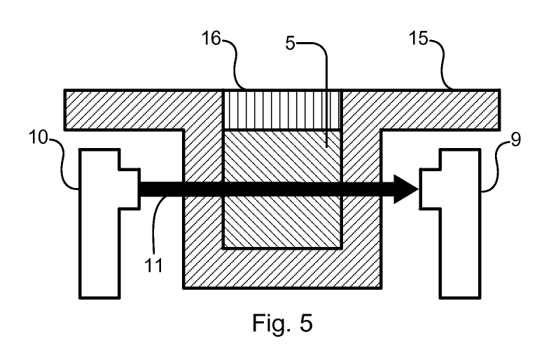

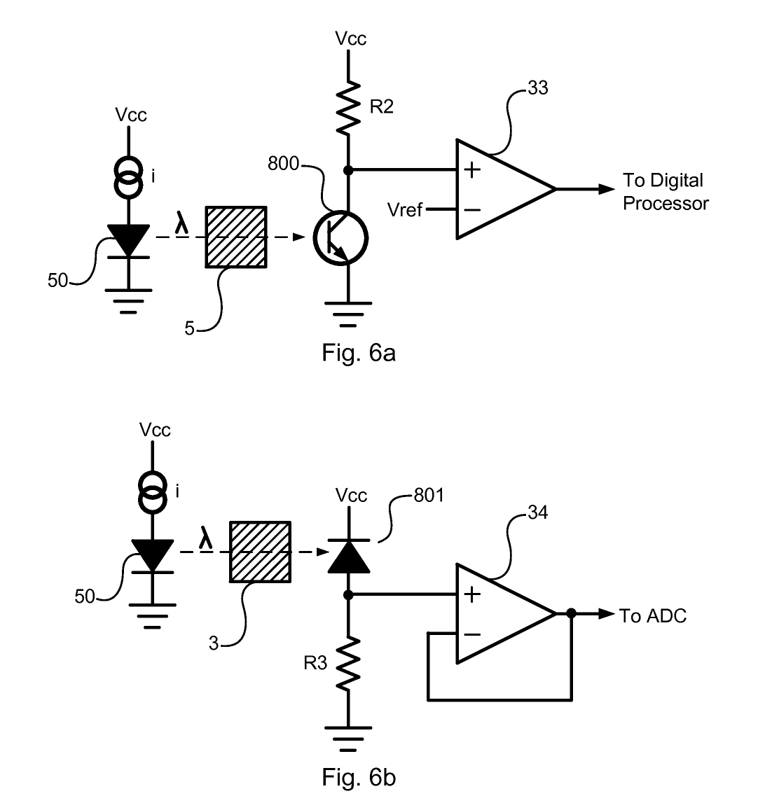

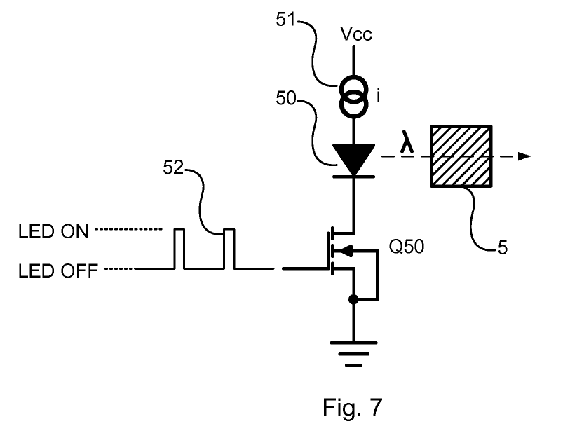

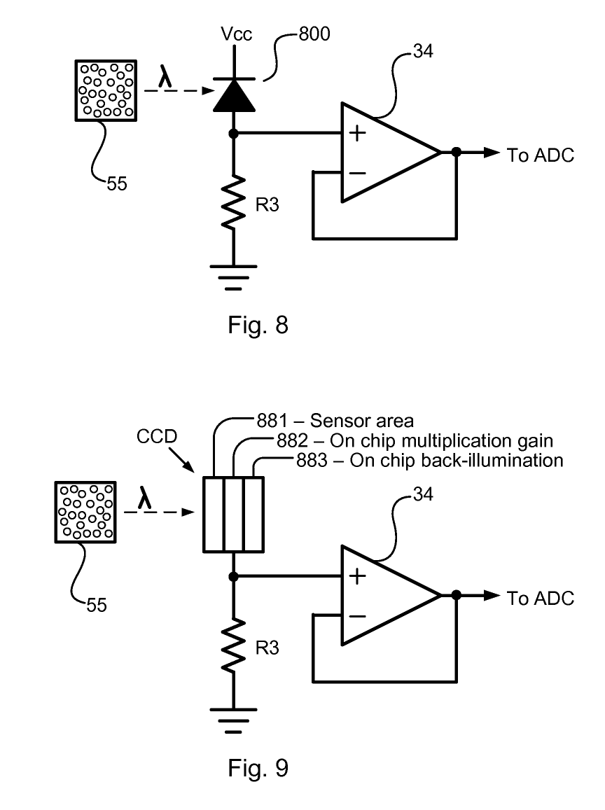

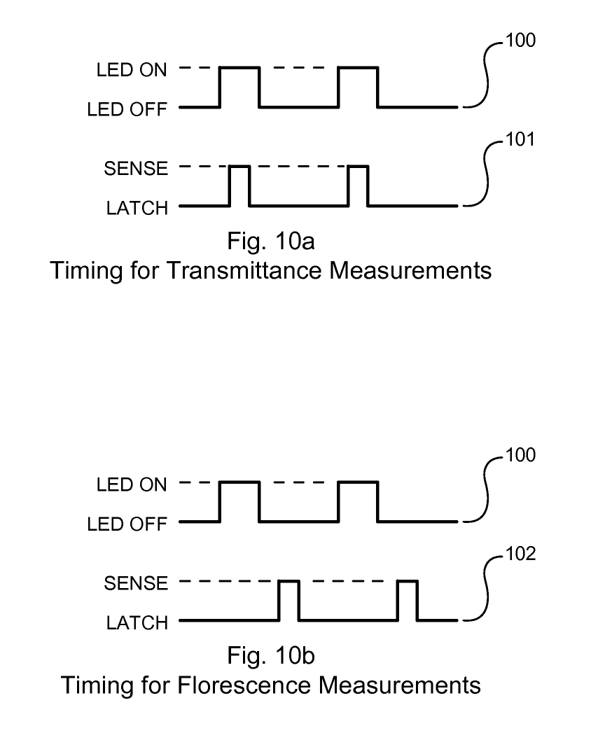

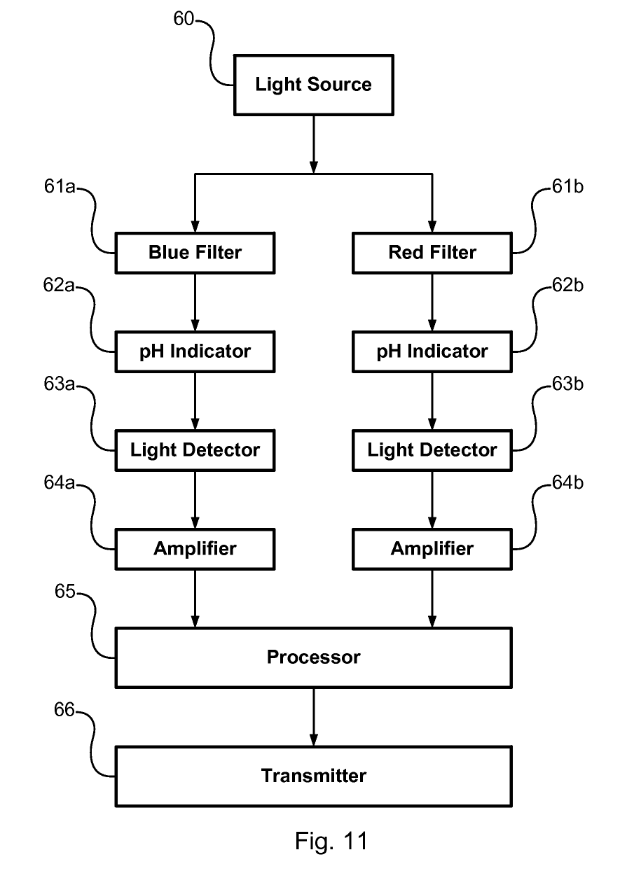

What is claimed is:

1. An ingestible diagnostic capsule comprising:
at least one well that consists material that changes optical properties in the presence of an analyte,
a means to measure the optical changes,
a means to transmit the measurements, the measurements corresponding to the presence of analytes in the digestive tract,
and a means to trigger an external image capture to indicate the position of the diagnostic capsule.

2. The apparatus of claim 1, wherein material in at least one well dissolves in the presence of specific enzymatic analytes.

3. The apparatus of claim 1, wherein at least one well is enteric coated so that embedded material is protected from the gastric environment.

4. The apparatus of claim 1, wherein material in at least one well changes color with the change of pH, the pH being determined by matching a vector table against the amplitudes of different wavelengths, the amplitudes being measured in separate wells.

5. The apparatus of claim 1, wherein at least one well is hollow so that the optical properties of surrounding fluid can be measured to characterize the presence of analyte.

6. The apparatus of claim 1, wherein the material of at least one well is a biosensor.

7. The apparatus of claim 1, wherein the material of at least one well is illuminated with a pulsating LED, the pulsation used to simultaneously increase measurement sensitivity while conserving power.

8. A general purpose ingestible diagnostic capsule comprising:
at least one hollow well,
at least one circuit that measures the optical properties of material inside the well,
at least one programmable system on chip that that can be programmed,
at least one means to transmit data that corresponds to the optical changes of the material,
and at least one means to trigger an image capture.

9. A method of determining the position of a diagnostic capsule within a digestive tract, the method comprising:
a means to collect data transmitted from the capsule,
a means to generate triggers to capture images,
a means to capture images when triggered,
a means to store the captured images that were captured from the triggers,
and a means to match collected data with the captured images.

10. The method of claim 9, wherein the means to capture images is an x-ray machine.

11. The method of claim 9, wherein the means to capture images is an ultrasonic imaging machine.

12. The method of claim 9, wherein the means to capture images is an antenna array system.

13. A method of determining the bioavailability of a substance, the method comprising a diagnostic pill used to transmit the optical properties of a surrounding fluid, the properties reflecting the presence of the substance.

14. A method of characterizing intestinal permeability, the method comprising:
at least one ingestible permeability marker,
a diagnostic capsule that can measure the concentration of the permeability marker,
a means to collect data from the diagnostic capsule,
a means to capture images that disclose the position of the capsule,
and a means to match images with collected data.
